SEQUENCE LISTING (23 sequences)

SEQ ID NO: 1 - protein, 165 aa, Artificial Sequence
  VEGF165 polypeptide, V165
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 2 - protein, 165 aa, Artificial Sequence
  VEGF165b polypeptide , V165b
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRSLTRKD

SEQ ID NO: 3 - protein, 121 aa, Artificial Sequence
  VEGF121 polypeptide, V121
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

SEQ ID NO: 4 - protein, 145 aa, Artificial Sequence
  VEGF145 polypeptide, V145
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVCDKPRR

SEQ ID NO: 5 - protein, 148 aa, Artificial Sequence
  VEGF148 polypeptide, V148
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKM

SEQ ID NO: 6 - protein, 183 aa, Artificial Sequence
  VEGF183 polypeptide, V183
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRPCGPCSERRKHLFVQDPQTCKCSCKNTDSRCKARQLELNERTCRCDKPRR

SEQ ID NO: 7 - protein, 189 aa, Artificial Sequence
  VEGF189 polypeptide, V189
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 8 - protein, 206 aa, Artificial Sequence
  VEGF206 polypeptide, V206
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 9 - protein, 28 aa, Artificial Sequence
  CTP polypeptide
SSSSKAPPPSLPSPSRLPGPSDTPILPQ

SEQ ID NO: 10 - protein, 358 aa, Artificial Sequence
  V165-CTP-V165 polypeptide
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRRSSSSKAPPPSLPSPSRLPGPSDTPILPQAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 11 - DNA, 363 bp, Artificial Sequence
  nucleotide sequence of VEGF121 gene
gccccgatggcggaaggtggtggtcaaaaccatcacgaggtagtcaaatttatggacgtttaccagcgctcttattgccacccaatcgaaacgctggttgatattttccaggaatatccggatgaaatcgaatacattttcaaaccgtcttgtgtcccactgatgcgctgtggtggctgctgcaatgacgagggcctggagtgcgttccaaccgaagaatccaatattacgatgcaaattatgcgtattaaaccgcaccaaggccaacacatcggtgaaatgtctttcctgcagcacaacaaatgtgaatgtcgcccgaagaaagaccgtgcacgccaggaaaagtgtgacaagccgcgtcgt

SEQ ID NO: 12 - DNA, 435 bp, Artificial Sequence
  nucleotide sequence of VEGF145 gene
gcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtgtgtgacaagccgaggcgg

SEQ ID NO: 13 - DNA, 444 bp, Artificial Sequence
  nucleotide sequence of VEGF148 gene
gcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaagatg

SEQ ID NO: 14 - DNA, 495 bp, Artificial Sequence
  nucleotide sequence of VEGF165 gene
gcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcgg

SEQ ID NO: 15 - DNA, 495 bp, Artificial Sequence
  nucleotide sequence of VEGF165b gene
gcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatctctcaccaggaaagac

SEQ ID NO: 16 - DNA, 549 bp, Artificial Sequence
  nucleotide sequence of VEGF183 gene
gcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccgtccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcgg

SEQ ID NO: 17 - DNA, 567 bp, Artificial Sequence
  nucleotide sequence of VEGF189 gene
gcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgttccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcgg

SEQ ID NO: 18 - DNA, 618 bp, Artificial Sequence
  nucleotide sequence of VEGF206 gene
gcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaaaaatcagttcgaggaaagggaaaggggcaaaaacgaaagcgcaagaaatcccggtataagtcctggagcgtgtacgttggtgcccgctgctgtctaatgccctggagcctccctggcccccatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcgg

SEQ ID NO: 19 - DNA, 84 bp, Artificial Sequence
  nucleotide sequence of CTP gene
tcctcttcctcaaaggcccctccccccagccttccaagtccatcccgactcccggggccctcggacaccccgatcctcccacaa

SEQ ID NO: 20 - DNA, 1077 bp, Artificial Sequence
  nucleotide sequence of V165-CTP-V165 gene
gcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtcctcttcctcaaaggcccctccccccagccttccaagtccatcccgactcccggggccctcggacaccccgatcctcccacaagcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtga

SEQ ID NO: 21 - RNA, 495 nt, Artificial Sequence
  nucleotide sequence of VEGF165 mRNA
gcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaucccugugggccuugcucagagcggagaaagcauuuguuuguacaagauccgcagacguguaaauguuccugcaaaaacacagacucgcguugcaaggcgaggcagcuugaguuaaacgaacguacuugcagaugugacaagccgaggcgg

SEQ ID NO: 22 - RNA, 84 nt, Artificial Sequence
  nucleotide sequence of CTP mRNA
uccucuuccucaaaggccccuccccccagccuuccaaguccaucccgacucccggggcccucggacaccccgauccucccacaa

SEQ ID NO: 23 - RNA, 1077 nt, Artificial Sequence
  nucleotide sequence of V165-CTP-V165 mRNA
gcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaucccugugggccuugcucagagcggagaaagcauuuguuuguacaagauccgcagacguguaaauguuccugcaaaaacacagacucgcguugcaaggcgaggcagcuugaguuaaacgaacguacuugcagaugugacaagccgaggcgguccucuuccucaaaggccccuccccccagccuuccaaguccaucccgacucccggggcccucggacaccccgauccucccacaagcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaucccugugggccuugcucagagcggagaaagcauuuguuuguacaagauccgcagacguguaaauguuccugcaaaaacacagacucgcguugcaaggcgaggcagcuugaguuaaacgaacguacuugcagaugugacaagccgaggcgguga

(19) United States
(12) Patent Application Publication
(10) Pub. No.: US 2023/0220026 A1
(43) Pub. Date: Jul. 13, 2023

(54) NUCLEIC ACID CARRIER FOR PRODUCING THE SINGLE-CHAIN VEGF FUSION PROTEIN WITH HIGH PHYSIOLOGICAL STABILITY AND DIMERIC EFFICIENCY, PREPARATION METHOD THEREOF, AND USE THEREOF

(71) Applicant: National Yang Ming Chiao Tung University, Taipei (TW)

(72) Inventors: Ching-Wei LUO, Taipei City (TW); Chi-Ying CHEN, Taipei City (TW)

(21) Appl. No.: 17/573,969

(22) Filed: Jan. 12, 2022

(51) Int. Cl. C07K 14/475; A61P 9/10; C12N 15/62

(52) U.S. Cl. CPC C07K 14/475; A61P 9/10; C12N 15/62; A61K 38/00

(57) ABSTRACT

The current invention provides the nucleic acid carrier for producing the single-chain VEGF fusion protein comprising the same, and methods of making and using the same.

### FIELD OF THE INVENTION

The present invention relates to a fusion protein as a pharmaceutical composition. In particular, the present invention relates to the nucleic acid carrier for producing the single-chain VEGF fusion protein with high physiological stability and dimeric efficiency.

### BACKGROUND OF THE INVENTION

Vascular endothelial-derived growth factors (VEGFs) are mitogen factors to regulate the processes of vasculogenesis and angiogenesis. Members of this family, including VEFG-A, VEGF-B, VEGF-C, VEGF-D, and VEGF-E, need to undergo dimerization between two protein subunits in order to be functional. The genes in each family can also generate multiple isoforms with different characteristics via alternative splicing. Among them, VEGF165 is the predominant isoform with potent bioactivity. It can act as a mitogen factor that primarily targets endothelial cells to maintain cell survival and promote proliferation. Herein, being a powerful signaling molecule, VEGF165 has been considered a potential protein drug capable of being applied in two directions, wound repair and cardiovascular regenerative therapeutics.

Effective wound healing requires neovascularization of the newly formed tissue. Delivery of angiogenic growth factors to injured or ischemic tissues to promote the targeted formation of new blood vessels has been proposed as an alternative approach to surgical revascularization procedures. Also, there is a growing interest in reactivating endogenous regenerative capacity locally by delivering angiogenic proteins directly or generating angiogenic proteins in situ. Bypassing the cellular transplantation, these strategies that directly manipulate the repair mechanisms locally open a new avenue of regenerative medicine that compensates for the inability of the heart to regenerate in adults. As a powerful angiogenic growth factor, VEGF165 can increase the density of blood vessels by promoting migration and proliferation of pre-existing endothelial cells and by increasing the vascular permeability to allow the efflux of inflammatory cells into the site of injury. Herein, VEGF165 represents a promising pharmaceutical protein to treat peripheral ischemia or diseases involving myocardial ischemia, such as myocardial infarction, heart failure, and stroke. Methods for promoting revascularization by VEGF165 in the heart have been conducted in multiple strategies, including injection of recombinant protein, non-viral or viral plasmid, and modified RNA

However, strategies mentioned above have doubts about the inconsistency of curative efficacy between trials, mainly due to the physiological instability of the functional protein. This result reflects the challenges of most therapeutic proteins, including instability in storage, short physiological half-life, immunogenicity, and synthesizing difficulty due to their structural complexity. For example, as far as physiological stability is concerned, clinical administration of the peptide and protein therapeutics shows fast degradation in bodies. Therefore, high-dosage administration and/or repetitive injection are required to maintain a therapeutic concentration for the course of treatment. However, the fluctuating concentration of a therapeutic protein with a high initial peak often causes side effects, including adverse inflammation and antagonists' expression. Not only this, there is another concern regarding the generation efficiency for VEGFs since these proteins require to form a dimeric structure in order to be functional. Therefore, as protein drugs, VEGFs face the problems of unpredictable pharmacokinetic action after being delivered to the subjects due to the low physiological stability and low dimeric efficiency.

In summary, the difficulty of recombinant VEGF165 protein in achieving a therapeutically relevant and durable dose for a lasting effect should be noticed. There is a need to optimize VEGFs for clinical application regarding their instability and uncertainty of structural properties issues in physiological conditions. The present invention addresses these and other requirements by providing a single-chain VEGF with improved bioavailability.

### SUMMARY OF THE INVENTION

An object of the invention is to develop a novel protein fusion method, which would not affect protein itself, and direct modification on the protein itself to improve pharmacokinetics properties.

The present invention provides a strategy to apply a linker peptide to link the monomers of candidate peptides, aiming not only to increase the physiological stability of the fusion protein but also to enhance their dimeric efficiency.

Another aspect of the present invention provides a nucleotide construct to produce single-chain VEGF molecules.

Detailed description of the invention is given in the following embodiments with reference to the accompanying drawings.

### BRIEF DESCRIPTION OF THE DRAWINGS

The foregoing summary, as well as the following detailed description of the invention, will be better understood when reading in conjunction with the appended drawings.

FIG. 1A is a schematic diagram of three constructs established for the VEGF-A bioactivity tests: V165, V165-CTP, and single-chain VEGF165 (V165-CTP-V165). For V165 expressing construct, the gene encoding the mature region of human VEGF165 was cloned. For V165-CTP expressing construct, the sequence encoding polypeptide CTP is added to 3′-end of the mature VEGF165 gene. For single-chain VEGF165 (V165-CTP-V165), two mature VEGF165 genes are linked by the sequence encoding CTP. Each construct was led by a signal peptide followed by a tag.

FIG. 1B shows the plasmid map of single-chain VEGF165 (V165-CTP-V165).

FIG. 1C shows the purification and characterization of the single-chain VEGF165 protein, including Coomassie blue staining (Left) and immunoblotting (Right) by using the antibody against VEGFA (GeneTex). The arrow indicates the position of single-chain VEGF165.

FIG. 2 depicts the protein efficacy of rhVEGF and V165-CTP-V165 by using luciferase reporter assay. HEK-293T cells with reporters and VEGFR2 were treated with rhVEGF purchased from R&D Systems, and purified 293T-expressed single-chain VEGF165. The luciferase results are expressed as mean±SD of triplicate determinations.

FIG. 3A-3C depicts that single-chain VEGF165 shows prolonged physiological stability in serum in vitro. PBS containing rhVEGF (R&D) or single-chain VEGF165 at 0.5 μg/ml concentration was used for immobilization on a 96-well plate. Mouse serum (pure) (FIG. 3A), rat serum (1:4 dilution with PBS) (FIG. 3B), and human serum (1:4 dilution with PBS, purchased from Valley Biomedical) (FIG. 3C) were added and incubated at indicated time intervals. The remaining VEGF proteins were detected using the antibody against VEGFA. The signal was determined by measuring the luminescent light intensity. The results are expressed as mean±SD of triplicate determinations. *P<0.05, **P<0.01, ***P<0.001 compared with rhVEGF (R&D).

FIG. 4A-4B depicts that single-chain VEGF165 exists prolonged serum half-life in vivo. Mice were injected with 1.5 μg of rhVEGF (n=3) or single-chain VEGF165 (n=4) dissolved in PBS intravenously. The blood samples were drawn at the indicated times. Serum levels of rhVEGF (R&D) (FIG. 4A) or single-chain VEGF165 (FIG. 4B) were determined by ELISA. Recombinant proteins were detected using the antibody against VEGFA (R&D), and the signal was determined by measuring the luminescent light intensity. The results are expressed as mean±SD of triplicate determinations.

FIG. 5A-5H illustrates that single-chain VEGF165 stimulates angiogenesis by Matrigel plug assay in vivo. C57BL/6 mice were injected subcutaneously on the inguinal region with Matrigel containing heparin (1.4 μg/ml) and single-chain VEGF165 or rhVEGF (R&D). PBS was used as the negative control (FIG. 5A). 250 μl of Matrigel containing rhVEGF (R&D) (100 ng/ml) or single-chain VEGF165 (100 ng/ml) were injected, and mice were sacrificed after 7 days. Matrigel plugs were removed, photographed, followed by histological analysis (FIG. 5B). Quantitative comparison of the number of cells per field in the plugs was shown (FIG. 5 C). For a longer duration, Matrigel containing rhVEGF (R&D) (200 ng/ml) or single-chain VEGF165 (200 ng/ml) were injected, and mice were sacrificed after 14 days. The Matrigel plugs were removed and photographed (FIG. 5D). Hematoxylin and eosin (H&E) staining of control group (FIG. 5E) and single-chain VEGF165 group (FIG. 5F) were performed. The arrow indicates the position of vascular formation (FIG. 5F). After 14 days of incubation, the degree of angiogenesis was determined by measuring the content of hemoglobin (OD 540 nm) of the plugs. Data are expressed as means±SD (FIG. 5G). Matrigel plugs were collected from different mice (n=8 in each group of different treatments), and the degree of angiogenesis was determined by measuring the content of hemoglobin (OD 540 nm). Data are expressed as means±SEM (FIG. 5H).

FIG. 6A-6B illustrates that single-chain VEGF165 promotes proliferation of endothelial cells. HMEC1 (2×10⁴ cells) were seeded in a 24-well plate. After overnight incubation, cells were then treated with 1 nM VEGF165 (R&D) or 1 nM purified single-chain VEGF165 in serum-free medium supplemented with BrdU. After 24 hours of incubation, cells were then fixed to detect BrdU by immunocytochemistry (ICC) (FIG. 6A). The ratio of BrdU positive cells per field was analyzed by the software Metamorph with Multi-Wavelength Cell Scoring Modules (FIG. 6B).

FIG. 7A-7E illustrates that single-chain VEGF165 promotes migration of endothelial cells. HMEC1 (1.5×10⁵ cells) were seeded on each side of an ibidi culture insert in a 12 well plate. After overnight incubation, the insert was gently removed. Cells were then treated with 1 nM VEGF (R&D), 1 nM, or 3 nM purified single-chain VEGF165 in serum-free medium. Cell motility was determined by photography after 24 hours of incubation. Images were recorded by light microscopy (FIG. 7A). The rate of migration for endothelial cells without treatment (FIG. 7B) or treated with 1 nM VEGF (R&D) (FIG. 7C), 1 nM (FIG. 7D), or 3 nM (FIG. 7E) purified single-chain VEGF165 was assessed by ImageJ and compared with respect to the area at 0 hour.

FIG. 8A-8F illustrates that single-chain VEGF165 promotes tube formation of endothelial cells. HUVEC cells (0.5×10⁵ cells) (FIG. 8A-8C) and HMEC1 (0.8×10⁵ cells) (FIG. 8D-8F) were mixed with 1 nM VEGF (R&D), conditioned medium, or purified single-chain VEGF165 and loaded into a Matrigel-coated 48 well plate. After 24 hours of incubation, tubes were stained by calcein-AM for 30 minutes. Representative pictures, the quantitative data of the number of nodes (i.e., branch points) (FIG. 8B, FIG. 8E), and total tube length (FIG. 8C, FIG. 8F) were shown. Images were recorded by fluorescence microscopy (FIG. 8A, FIG. 8D) and analyzed by the software Metamorph with Angiogenesis Application Modules.

FIG. 9A-9D illustrates that the therapeutic effects of single-chain VEGF165 on wound healing are much better than those of rhVEGF. FIG. 9A illustrates representative photographs of the status of wound repair. Two pairs of 4-mm full-thickness excisional wounds on the dorsal skin were generated (Day 0). Each wound was treated with a single dose of 100 ng rhVEGF (R&D) or single-chain VEGF165 on day 0. Digital photographs were taken on day 0, day 4, and day 8. FIG. 9B-9C illustrates the quantification of wound healing on day 4 and day 8. Percentage of wound area closed (FIG. 9B), and the diameter of the wound (FIG. 9C) was shown. *P<0.05, **P<0.01 compared with the rhVEGF (R&D) group. Photomicrographs of HE-stained histologic sections of wound site skin at Day 4 and Day 8 (100×)(FIG. 9D).

FIG. 10 demonstrates that single-chain VEGF165 treatment exhibits a rapid epithelialization and matrix reorganization of wound healing better than rhVEGF. Immunohistochemical analysis of wound site skin at Day 8 with antibodies against Keratin 5 (K5) and Ki67. Wound site skin treated with single-chain VEGF165 shows intense epidermis (K5) and dermis remodeling as compared with that treated with PBS or rhVEGF. Ki67 for cell proliferation was found in the basal layer of skin epithelial and the matrix region of dermis.

### DETAILED DESCRIPTION OF THE INVENTION

While preferred embodiments of the invention are shown and described herein, such embodiments are provided by way of example only and are not intended to limit the scope of the invention otherwise. Various alternatives to the described embodiments of the invention may be employed in practicing the invention.

Unless otherwise defined, all technical terms used herein have the same meaning as commonly understood by one of ordinary skill in the art to which this invention belongs.

The present invention provides a nucleic acid carrier comprising a first polynucleotide, a linker polynucleotide and a second polynucleotide for producing a fusion protein or a derived mRNA. The first polynucleotide and the second polynucleotide is selected from a VEGF gene, whereas the linker polynucleotide is selected from the CTP gene.

Optionally, in an exemplary embodiment of the present invention, the VEGF gene of the present invention includes, but is not limited to, VEGF165 gene (SEQ ID NO: 14), VEGF165b gene (SEQ ID NO: 15), VEGF121 gene (SEQ ID NO: 11), VEGF145 gene (SEQ ID NO: 12), VEGF148 gene (SEQ ID NO: 13), VEGF183 gene (SEQ ID NO: 16), VEGF189 gene (SEQ ID NO: 17), and VEGF206 gene (SEQ ID NO: 18).

The VEGF mRNA is derived from the VEGF gene. For example, in some embodiments, the VEGF mRNA in the present invention includes, but is not limited to, VEGF165 mRNA (SEQ ID NO:21), VEGF165b mRNA, VEGF121 mRNA, VEGF145 mRNA, VEGF148 mRNA, VEGF183 mRNA, VEGF189 mRNA, and VEGF206 mRNA.

On the other side, the VEGF polypeptide is encoded by the VEGF gene. The VEGF polypeptide in the present invention includes, but is not limited to, VEGF165 polypeptide (SEQ ID NO: 1), VEGF165b polypeptide (SEQ ID NO: 2), VEGF121 polypeptide (SEQ ID NO: 3), VEGF145 polypeptide (SEQ ID NO: 4), VEGF148 polypeptide (SEQ ID NO: 5), VEGF183 polypeptide (SEQ ID NO: 6), VEGF189 polypeptide (SEQ ID NO: 7), and VEGF206 polypeptide (SEQ ID NO: 8).

In addition, the linker polynucleotide includes, but is not limited to, the CTP gene (SEQ ID NO: 19). A CTP mRNA (SEQ ID NO:22) and a encoded CTP polypeptide (SEQ ID NO: 9) is derived from the CTP gene (SEQ ID NO: 19).

In one embodiment, the fusion protein includes, but is not limited to, V165-CTP-V165 polypeptide (SEQ ID NO: 10). In another embodiment, the derived mRNA includes, but is not limited to, V165-CTP-V165 mRNA (SEQ ID NO: 23).

Sequences that can be employed in accordance with the invention are shown herein below:

SEQ ID NO: 1 (VEGF165 polypeptide, V165):
APMAEGGGQNHHEVVKFMDVYQRSYCHPIETLVDIFQEYP
DEIEYIFKPSCVPLMRCGGCCNDEGLECVPTEESNITMQI
MRIKPHQGQHIGEMSFLQHNKCECRPKKDRARQENPCGPC
SERRKHLFVQDPQTCKCSCKNTDSRCKARQLELNERTCRC
DKPRR
SEQ ID NO: 2 (VEGF165b polypeptide,
V165b):
APMAEGGGQNHHEVVKFMDVYQRSYCHPIETLVDIFQEYP
DEIEYIFKPSCVPLMRCGGCCNDEGLECVPTEESNITMQI
MRIKPHQGQHIGEMSFLQHNKCECRPKKDRARQENPCGPC
SERRKHLFVQDPQTCKCSCKNTDSRCKARQLELNERTCRS
LTRKD
SEQ ID NO: 3 (VEGF121 polypeptide, V121):
APMAEGGGQNHHEVVKFMDVYQRSYCHPIETLVDIFQEYP
DEIEYIFKPSCVPLMRCGGCCNDEGLECVPTEESNITMQI
MRIKPHQGQHIGEMSFLQHNKCECRPKKDRARQEKCDKPR
R
SEQ ID NO: 4 (VEGF145 polypeptide, V145):
APMAEGGGQNHHEVVKFMDVYQRSYCHPIETLVDIFQEYP
DEIEYIFKPSCVPLMRCGGCCNDEGLECVPTEESNITMQI
MRIKPHQGQHIGEMSFLQHNKCECRPKKDRARQEKKSVRG
KGKGQKRKRKKSRYKSWSVCDKPRR
SEQ ID NO: 5 (VEGF148 polypeptide, V148):
APMAEGGGQNHHEVVKFMDVYQRSYCHPIETLVDIFQEYP
DEIEYIFKPSCVPLMRCGGCCNDEGLECVPTEESNITMQI
MRIKPHQGQHIGEMSFLQHNKCECRPKKDRARQENPCGPC
SERRKHLFVQDPQTCKCSCKNTDSRCKM
SEQ ID NO: 6 (VEGF183 polypeptide, V183):
APMAEGGGQNHHEVVKFMDVYQRSYCHPIETLVDIFQEYP
DEIEYIFKPSCVPLMRCGGCCNDEGLECVPTEESNITMQI
MRIKPHQGQHIGEMSFLQHNKCECRPKKDRARQEKKSVRG
KGKGQKRKRKKSRPCGPCSERRKHLFVQDPQTCKCSCKNT
DSRCKARQLELNERTCRCDKPRR
SEQ ID NO: 7 (VEGF189 polypeptide, V189):
APMAEGGGQNHHEVVKFMDVYQRSYCHPIETLVDIFQEYP
DEIEYIFKPSCVPLMRCGGCCNDEGLECVPTEESNITMQI
MRIKPHQGQHIGEMSFLQHNKCECRPKKDRARQEKKSVRG
KGKGQKRKRKKSRYKSWSVPCGPCSERRKHLFVQDPQTCK
CSCKNTDSRCKARQLELNERTCRCDKPRR
SEQ ID NO: 8 (VEGF206 polypeptide, V206):
APMAEGGGQNHHEVVKFMDVYQRSYCHPIETLVDIFQEYP
DEIEYIFKPSCVPLMRCGGCCNDEGLECVPTEESNITMQI
MRIKPHQGQHIGEMSFLQHNKCECRPKKDRARQEKKSVRG
KGKGQKRKRKKSRYKSWSVYVGARCCLMPWSLPGPHPCGP
CSERRKHLFVQDPQTCKCSCKNTDSRCKA
RQLELNERTCRCDKPRR
SEQ ID NO: 9 (CTP polypeptide):
SSSSKAPPPSLPSPSRLPGPSDTPILPQ
SEQ ID NO: 10 (V165-CTP-V165 polypeptide):
APMAEGGGQNHHEVVKFMDVYQRSYCHPIETLVDIFQEYP
DEIEYIFKPSCVPLMRCGGCCNDEGLECVPTEESNITMQI
MRIKPHQGQHIGEMSFLQHNKCECRPKKDRARQENPCGPC
SERRKHLFVQDPQTCKCSCKNTDSRCKARQLELNERTCRC
DKPRRSSSSKAPPPSLPSPSRLPGPSDTPILPQAPMAEGG
GQNHHEVVKFMDVYQRSYCHPIETLVDIFQEYPDEIEYIF
KPSCVPLMRCGGCCNDEGLECVPTEESNITMQIMRIKPHQ
GQHIGEMSFLQHNKCECRPKKDRARQENPCGPCSERRKHL
FVQDPQTCKCSCKNTDSRCKARQLELNERTCRCDKPRR
SEQ ID NO: 11 (nucleotide sequence of VE
GF121 gene):
gccccgatggcggaaggtggtggtcaaaaccatcacgagg
tagtcaaatttatggacgtttaccagcgctcttattgcca
cccaatcgaaacgctggttgatattttccaggaatatccg
gatgaaatcgaatacattttcaaaccgtcttgtgtcccac
tgatgcgctgtggtggctgctgcaatgacgagggcctgga
gtgcgttccaaccgaagaatccaatattacgatgcaaatt
atgcgtattaaaccgcaccaaggccaacacatcggtgaaa
tgtctttcctgcagcacaacaaatgtgaatgtcgcccgaa
gaaagaccgtgcacgccaggaaaagtgtgacaagccgcgt
cgt
SEQ ID NO: 12 (nucleotide sequence of VE
GF145 gene):
gcacccatggcagaaggaggagggcagaatcatcacgaag
tggtgaagttcatggatgtctatcagcgcagctactgcca
tccaatcgagaccctggtggacatcttccaggagtaccct
gatgagatcgagtacatcttcaagccatcctgtgtgcccc
tgatgcgatgcgggggctgctgcaatgacgagggcctgga
gtgtgtgcccactgaggagtccaacatcaccatgcagatt
atgcggatcaaacctcaccaaggccagcacataggagaga
tgagcttcctacagcacaacaaatgtgaatgcagaccaaa
gaaagatagagcaagacaagaaaaaaaatcagttcgagga
aagggaaaggggcaaaaacgaaagcgcaagaaatcccggt
ataagtcctggagcgtgtgtgacaagccgaggcgg
SEQ ID NO: 13 (nucleotide sequence of VE
GF148 gene):
gcacccatggcagaaggaggagggcagaatcatcacgaag
tggtgaagttcatggatgtctatcagcgcagctactgcca
tccaatcgagaccctggtggacatcttccaggagtaccct
gatgagatcgagtacatcttcaagccatcctgtgtgcccc
tgatgcgatgcgggggctgctgcaatgacgagggcctgga
gtgtgtgcccactgaggagtccaacatcaccatgcagatt
atgcggatcaaacctcaccaaggccagcacataggagaga
tgagcttcctacagcacaacaaatgtgaatgcagaccaaa
gaaagatagagcaagacaagaaaatccctgtgggccttgc
tcagagcggagaaagcatttgtttgtacaagatccgcaga
cgtgtaaatgttcctgcaaaaacacagactcgcgttgcaa
gatg
SEQ ID NO: 14 (nucleotide sequence of VE
GF165 gene):
gcacccatggcagaaggaggagggcagaatcatcacgaag
tggtgaagttcatggatgtctatcagcgcagctactgcca
tccaatcgagaccctggtggacatcttccaggagtaccct
gatgagatcgagtacatcttcaagccatcctgtgtgcccc
tgatgcgatgcgggggctgctgcaatgacgagggcctgga
gtgtgtgcccactgaggagtccaacatcaccatgcagatt
atgcggatcaaacctcaccaaggccagcacataggagaga
tgagcttcctacagcacaacaaatgtgaatgcagaccaaa
gaaagatagagcaagacaagaaaatccctgtgggccttgc
tcagagcggagaaagcatttgtttgtacaagatccgcaga
cgtgtaaatgttcctgcaaaaacacagactcgcgttgcaa
ggcgaggcagcttgagttaaacgaacgtacttgcagatgt
gacaagccgaggcgg
SEQ ID NO: 15 (nucleotide sequence of VE
GF165b gene):
gcacccatggcagaaggaggagggcagaatcatcacgaag
tggtgaagttcatggatgtctatcagcgcagctactgcca
tccaatcgagaccctggtggacatcttccaggagtaccct
gatgagatcgagtacatcttcaagccatcctgtgtgcccc
tgatgcgatgcgggggctgctgcaatgacgagggcctgga
gtgtgtgcccactgaggagtccaacatcaccatgcagatt
atgcggatcaaacctcaccaaggccagcacataggagaga
tgagcttcctacagcacaacaaatgtgaatgcagaccaaa
gaaagatagagcaagacaagaaaatccctgtgggccttgc
tcagagcggagaaagcatttgtttgtacaagatccgcaga
cgtgtaaatgttcctgcaaaaacacagactcgcgttgcaa
ggcgaggcagcttgagttaaacgaacgtacttgcagatct
ctcaccaggaaagac
SEQ ID NO: 16 (nucleotide sequence of VE
GF183 gene):
gcacccatggcagaaggaggagggcagaatcatcacgaag
tggtgaagttcatggatgtctatcagcgcagctactgcca
tccaatcgagaccctggtggacatcttccaggagtaccct
gatgagatcgagtacatcttcaagccatcctgtgtgcccc
tgatgcgatgcgggggctgctgcaatgacgagggcctgga
gtgtgtgcccactgaggagtccaacatcaccatgcagatt
atgcggatcaaacctcaccaaggccagcacataggagaga
tgagcttcctacagcacaacaaatgtgaatgcagaccaaa
gaaagatagagcaagacaagaaaaaaaatcagttcgagga
aagggaaaggggcaaaaacgaaagcgcaagaaatcccgtc
cctgtgggccttgctcagagcggagaaagcatttgtttgt
acaagatccgcagacgtgtaaatgttcctgcaaaaacaca
gactcgcgttgcaaggcgaggcagcttgagttaaacgaac
gtacttgcagatgtgacaagccgaggcgg
SEQ ID NO: 17 (nucleotide sequence of VE
GF189 gene):
gcacccatggcagaaggaggagggcagaatcatcacgaag
tggtgaagttcatggatgtctatcagcgcagctactgcca
tccaatcgagaccctggtggacatcttccaggagtaccct
gatgagatcgagtacatcttcaagccatcctgtgtgcccc
tgatgcgatgcgggggctgctgcaatgacgagggcctgga
gtgtgtgcccactgaggagtccaacatcaccatgcagatt
atgcggatcaaacctcaccaaggccagcacataggagaga
tgagcttcctacagcacaacaaatgtgaatgcagaccaaa
gaaagatagagcaagacaagaaaaaaaatcagttcgagga
aagggaaaggggcaaaaacgaaagcgcaagaaatcccggt
ataagtcctggagcgttccctgtgggccttgctcagagcg
gagaaagcatttgtttgtacaagatccgcagacgtgtaaa
tgttcctgcaaaaacacagactcgcgttgcaaggcgaggc
agcttgagttaaacgaacgtacttgcagatgtgacaagcc
gaggcgg
SEQ ID NO: 18 (nucleotide sequence of VE
GF206 gene):
gcacccatggcagaaggaggagggcagaatcatcacgaag
tggtgaagttcatggatgtctatcagcgcagctactgcca
tccaatcgagaccctggtggacatcttccaggagtaccct
gatgagatcgagtacatcttcaagccatcctgtgtgcccc
tgatgcgatgcgggggctgctgcaatgacgagggcctgga
gtgtgtgcccactgaggagtccaacatcaccatgcagatt
atgcggatcaaacctcaccaaggccagcacataggagaga
tgagcttcctacagcacaacaaatgtgaatgcagaccaaa
gaaagatagagcaagacaagaaaaaaaatcagttcgagga
aagggaaaggggcaaaaacgaaagcgcaagaaatcccggt
ataagtcctggagcgtgtacgttggtgcccgctgctgtct
aatgccctggagcctccctggcccccatccctgtgggcct
tgctcagagcggagaaagcatttgtttgtacaagatccgc
agacgtgtaaatgttcctgcaaaaacacagactcgcgttg
caaggcgaggcagcttgagttaaacgaacgtacttgcaga
tgtgacaagccgaggcgg
SEQ ID NO: 19 (nucleotide sequence of CT
P gene):
tcctcttcctcaaaggcccctccccccagccttccaagtc
catcccgactcccggggccctcggacaccccgatcctccc
acaa
SEQ ID NO: 20 (nucleotide sequence of V1
65-CTP-V165 gene):
gcacccatggcagaaggaggagggcagaatcatcacgaag
tggtgaagttcatggatgtctatcagcgcagctactgcca
tccaatcgagaccctggtggacatcttccaggagtaccct
gatgagatcgagtacatcttcaagccatcctgtgtgcccc
tgatgcgatgcgggggctgctgcaatgacgagggcctgga
gtgtgtgcccactgaggagtccaacatcaccatgcagatt
atgcggatcaaacctcaccaaggccagcacataggagaga
tgagcttcctacagcacaacaaatgtgaatgcagaccaaa
gaaagatagagcaagacaagaaaatccctgtgggccttgc
tcagagcggagaaagcatttgtttgtacaagatccgcaga
cgtgtaaatgttcctgcaaaaacacagactcgcgttgcaa
ggcgaggcagcttgagttaaacgaacgtacttgcagatgt
gacaagccgaggcggtcctcttcctcaaaggcccctcccc
ccagccttccaagtccatcccgactcccggggccctcgga
caccccgatcctcccacaagcacccatggcagaaggagga
gggcagaatcatcacgaagtggtgaagttcatggatgtct
atcagcgcagctactgccatccaatcgagaccctggtgga
catcttccaggagtaccctgatgagatcgagtacatcttc
aagccatcctgtgtgcccctgatgcgatgcgggggctgct
gcaatgacgagggcctggagtgtgtgcccactgaggagtc
caacatcaccatgcagattatgcggatcaaacctcaccaa
ggccagcacataggagagatgagcttcctacagcacaaca
aatgtgaatgcagaccaaagaaagatagagcaagacaaga
aaatccctgtgggccttgctcagagcggagaaagcatttg
tttgtacaagatccgcagacgtgtaaatgttcctgcaaaa
acacagactcgcgttgcaaggcgaggcagcttgagttaaa
cgaacgtacttgcagatgtgacaagccgaggcggtga
SEQ ID NO: 21 (nucleotide sequence of VE
GF165 mRNA):
gcacccauggcagaaggaggagggcagaaucaucacgaag
uggugaaguucauggaugucuaucagcgcagcuacugcca
uccaaucgagacccugguggacaucuuccaggaguacccu
gaugagaucgaguacaucuucaagccauccugugugcccc
ugaugcgaugcgggggcugcugcaaugacgagggccugga
gugugugcccacugaggaguccaacaucaccaugcagauu
augcggaucaaaccucaccaaggccagcacauaggagaga
ugagcuuccuacagcacaacaaaugugaaugcagaccaaa
gaaagauagagcaagacaagaaaaucccugugggccuugc
ucagagcggagaaagcauuuguuuguacaagauccgcaga
cguguaaauguuccugcaaaaacacagacucgcguugcaa
ggcgaggcagcuugaguuaaacgaacguacuugcagaugu
gacaagccgaggcgg
SEQ ID NO: 22 (nucleotide sequence of CT
P mRNA):
uccucuuccucaaaggccccuccccccagccuuccaaguc
caucccgacucccggggcccucggacaccccgauccuccc
acaa
SEQ ID NO: 23 (nucleotide sequence of V1
65-CTP-V165 mRNA):
gcacccauggcagaaggaggagggcagaaucaucacgaag
uggugaaguucauggaugucuaucagcgcagcuacugcca
uccaaucgagacccugguggacaucuuccaggaguacccu
gaugagaucgaguacaucuucaagccauccugugugcccc
ugaugcgaugcgggggcugcugcaaugacgagggccugga
gugugugcccacugaggaguccaacaucaccaugcagauu
augcggaucaaaccucaccaaggccagcacauaggagaga
ugagcuuccuacagcacaacaaaugugaaugcagaccaaa
gaaagauagagcaagacaagaaaaucccugugggccuugc
ucagagcggagaaagcauuuguuuguacaagauccgcaga
cguguaaauguuccugcaaaaacacagacucgcguugcaa
ggcgaggcagcuugaguuaaacgaacguacuugcagaugu
gacaagccgaggcgguccucuuccucaaaggccccucccc
ccagccuuccaaguccaucccgacucccggggcccucgga
caccccgauccucccacaagcacccauggcagaaggagga
gggcagaaucaucacgaaguggugaaguucauggaugucu
aucagcgcagcuacugccauccaaucgagacccuggugga
caucuuccaggaguacccugaugagaucgaguacaucuuc
aagccauccugugugccccugaugcgaugcgggggcugcu
gcaaugacgagggccuggagugugugcccacugaggaguc
caacaucaccaugcagauuaugcggaucaaaccucaccaa
ggccagcacauaggagagaugagcuuccuacagcacaaca
aaugugaaugcagaccaaagaaagauagagcaagacaaga
aaaucccugugggccuugcucagagcggagaaagcauuug
uuuguacaagauccgcagacguguaaauguuccugcaaaa
acacagacucgcguugcaaggcgaggcagcuugaguuaaa
cgaacguacuugcagaugugacaagccgaggcgguga

A subject may be a human being or a non-human animal, such as cat, dog, rabbit, cattle, horse, sheep, goat, monkey, mouse, rat, gerbil, guinea pig, pig, but is preferably a human.

The healing of a wound is a complex process that involves four major phases: hemostasis, inflammation, proliferation, and tissue remodeling. After the injury, hemostasis and the inflammatory response would first progress. In the next stage, fibroblastic cells would be attracted to the wound bed site and form granulation tissue. Concurrently, epithelial cells would migrate to the surface of the wound bed and proliferate to provide epithelial coverage, which is called re-epithelialization. During this stage, angiogenesis is a crucial process by which allows the efflux of cells, oxygen, and nutrition into the injury site to accelerate tissue repair. The late stage of healing will enter the maturation phase, in which granulation tissue would undergo programmed cell death, followed by collagenous matrix reorganization and restoration of the skin barrier.

The primary goal of wound therapy is to accelerate the healing process, where an adequate blood supply is needed to promote the granulation matrix. However, issues such as illness, living habits, nutrition state, stress, or medications would cause challenges to angiogenesis and thus delay wound healing.

A reduced wound size within the meaning of the present invention is defined as wound size at a certain time after the start of treatment according to the invention in % of the wound size at day 0 of treatment. Examples are a wound size at a certain time after the start of treatment of less than 90%, less than 80%, less than 70%, less than 60%, less than 50%, less than 40%, less than 30%, or less than 20% of the wound size at day 0 of treatment.

The reduced wound size (or wound area reduction, WAR) may be assessed within a timeframe of e.g., 0-8 days. The reduced wound size is considered a relevant parameter, indicating a treatment effect, since WAR is regarded as a reliable predictor of later complete wound closure (Cardinal M E, Harding K et al., Wound Rep Reg (2008) 16 19-22).

Within the meaning of the present invention, the term “reduced wound size” is used synonymously with the term “wound area reduction (WAR).”

The wound size (or wound area) can be determined by quantitative analysis, e.g., area measurements of the wound, diameter measurements of the wound, planimetric tracings of the wound, etc. Bright-field photographs of the wound, histological analysis of the wound, such as immunohistochemical staining, can also be used to assess wound healing.

The success of wound therapy is reflected by and may be assessed via an increased wound closure rate, a reduced wound size, a shorter time to wound closure, an increase of the re-epithelialization of the chronic wound, and a reduction of pain related to the chronic wound.

Epithelialization is an essential component of wound healing, used as a defining parameter of successful wound closure. Epithelialization is defined as a process of covering epithelial surface of the wound and forming a barrier breach against infection. A wound cannot be considered healed in the absence of re-epithelialization. The epithelialization process is impaired in all types of chronic wounds. Failure of epidermal keratinocytes proliferation and migration to maintain the epithelial barrier may contribute to wound reoccurrence, which is another significant clinical problem. A better understanding of the epithelialization process may provide insights for new therapeutic approaches to accelerate wound closure. (Epithelialization in Wound Healing: A Comprehensive Review, Pastar et al., ADVANCES IN WOUND CARE, 2014, VOLUME 3, NUMBER 7, 445-464)

The term “normal wound” refers to a wound that undergoes regular wound healing repair.

As used herein, the term “chronic wound” refers to a wound that has not healed within a normal time period for healing in an otherwise healthy subject. Chronic wounds maybe those that do not heal because of the health of the subject; for example, where the subject has poor circulation or a disease such as diabetes, or where the subject is on a medication that inhibits the normal healing process. In some instances, a chronic wound may remain unhealed for weeks, months, or even years. Examples of chronic wounds include but are not limited to diabetic ulcers, pressure sores, venous ulcers, and tropical ulcers.

The term “acute wound” is an injury to the tissue that occurs by primary intention or any traumatic or surgical wound. Acute wounds include, but are not limited to, wounds caused by thermal injury, trauma, surgery, excision of extensive skin cancer, deep fungal and bacterial infections, vasculitis, scleroderma, pemphigus, toxic epidermal necrolysis, etc. An acute wound can develop into a chronic wound.

Ischemia of the tissues of an arm or leg in the human, or a forelimb or hindlimb in a mammal, can also result from dissection or occlusion of an artery that supplies blood to those tissues. Ischemic cardiac tissue may be located by angiogram, while compromised circulation in the peripheral vasculature can be identified by other conventional techniques.

The term “ischemia” is an inadequate blood supply to a local area due to blockage of blood vessels. The present invention highlights that the application for the direct and local delivery of therapeutic protein at or near the location(s), typically ischemic cardiac or other tissue damage.

The present invention can also apply to the ischemic injury to the limb tissue, or the site required for a surgical procedure, or after the onset of ischemia, which may accompany a traumatic injury. The therapeutic protein or protein-encoding vector can be delivered intravenously, orally, parenterally, or by direct injection at the site of the ischemic injury, varying depending on different factors.

The present invention is also feasible for subjects undergoing treatments that might delay or provide ineffective wound healing. Treatments can include but are not limited to steroids medications, radiation therapy, and immune suppression, etc.

In one embodiment, a “pharmaceutical composition” in the present invention includes, but is not limited to, the fusion protein/the therapeutic protein or the derived mRNA. The fusion protein includes, but is not limited to, V165-CTP-V165 polypeptide (SEQ ID NO: 10). The derived mRNA includes, but is not limited to, V165-CTP-V165 mRNA (SEQ ID NO: 23).

Generally, the dosage required to provide an effective amount of a “pharmaceutical composition”, which can be adjusted by one skilled in the art, will vary depending on several factors, including the age, health, physical condition, weight, type, and extent of the disease or disorder of the recipient, frequency of treatment, the nature of concurrent therapy, if required, and the nature and scope of the desired effect(s) (Nies et al., Chapter 3 In: Goodman & Gilman's The Pharmacological Basis of Therapeutics, 9th Ed., Hardman et al., eds., McGraw-Hill, New York, N.Y., 1996).

Whilst the dosage of the pharmaceutical composition used will vary according to the activity and the condition being treated, it may be stated by way of guidance that a dosage selected in the range from 1 μg/cm² to 250 μg/cm²

### EXAMPLES

The embodiments encompassed herein are now described with reference to the following examples. These examples are provided for the purpose of illustration only, and the disclosure encompassed herein should in no way be construed as being limited to these examples, but rather should be construed to encompass any and all variations which become evident as a result of the teachings provided herein.

Each treatment condition was performed in at least triplicate in an experiment. For in vivo assays, mouse numbers used in each group were indicated. Data were presented as means±SD. For data collected from at least three independent experiments, the statistical results were shown as means±SEM. For representative images, at least three independent experiments showed similar results. For comparison between two groups, the Student's t-test was used. For comparison of multiple groups, one-way ANOVA followed by Bonferroni posttest was used. *<0.05, **<0.01, ***<0.001.

### Example 1

Construction of Single-Chain VEGF165 Expression Plasmid

The fragment of human VEGF165 and CTP can be synthesized de novo or by using the polymerase chain reaction (PCR) method. The fidelity of each DNA product was further confirmed by DNA sequencing before using in expression studies. In the following, the recombinant fragments were cloned into the expression vector pcDNA3.1 for mammalian expression constructs. In various embodiments, any expression vector known to the skilled artisan that is compatible with a mammalian expression system can be used. As used herein, the term “expression” means transcription and/or translation of nucleic acid in a host cell. A mammalian expression system may include, but is not limited to, the mammalian host cell and the expression vector used to express the protein. The expression vector can contain a promoter described herein, and the promoter is heterologous to the expression vector. In one embodiment, secretion of the protein into the culture medium is controlled by a signal peptide (e.g., Prolactin signal peptide) incorporated into the expression vector and which is capable of directing the secretion of the expressed protein out of the mammalian cell. In other embodiments, other signal peptides suitable for facilitating the secretion of the protein are known to those skilled in the art. In one aspect, the signal peptide is typically cleaved from the protein after secretion.

### Example 2

Generation of Polynucleotides Encoding V165-CTP-V165

The fragment of the single-chain VEGF165 expression plasmid will be used as the template for generating polynucleotides. Polynucleotides encoding V165-CTP-V165 were synthesized in vitro by T7 RNA polymerase-mediated transcription from a linearized DNA template, which incorporates UTRs and a poly-A tail region. In various embodiments, any expression vector and RNA polymerase known to the skilled artisan that is compatible with a mammalian expression system can be used. In some embodiments, a donor methyl group S-adenosylmethionine (SAM) could be added to the methylated capped RNA to increase mRNA translation efficiency. Previous studies have described that there are now known to be more than 100 modifications of these bases, some of which affect transcript stability and can cause alternative folding, leading to new tertiary structures. For example, replacing pseudouridine with 1-methylpseudouridine (m1J) or N1-methylpseudouridine, and further optimizations in purification and capping efficiency would stabilize mRNA transcripts and increase the translation level without causing an innate immune response.

After purification, the mRNA was diluted in the corresponding buffer to the desired concentration. The “purification” of the nucleic acids described herein are capable of specific hybridization, under appropriate hybridization conditions (e.g., appropriate buffer, ionic strength, temperature), to a complementary nucleic acid. Techniques for synthesizing the purified nucleic acids described herein are well-known in the art and include chemical syntheses and recombinant methods. Purified nucleic acid molecules can also be made commercially. The purified nucleic acids can be analyzed by techniques known in the art, such as restriction enzyme analysis or sequencing, to determine the sequence of the isolated nucleic acids. The purified polynucleotides in the appropriate buffer can be directly injected into the wound site area, which varies on the cases (e.g., intramyocardial injection in myocardial infarction model).

### Example 3

Expression of Recombinant Human V165-CTP-V165 Protein

Mammalian cells are transfected with an expression vector comprising V165-CTP-V165 encoding the protein by using procedures well-known to those skilled in the art. The term “transfection” means transferring a nucleic acid, or a nucleic acid fragment, into a host cell via a non-viral approach. Such transfection protocols include calcium phosphate, cationic lipid or polymers, hydrodynamic delivery method, and the use of electroporation. The mammalian expression system can be established by any cell line that is known to the skilled artisan. In various embodiments, the transfected mammalian cells may be grown by techniques including batch, flask, or Patri-dish in the corresponding medium supplemented with FBS. Stable cell lines were selected under specific centration of antibiotics for consistent production of the recombinant proteins. The conditioned medium was collected and verified by molecular-cell-based methods.

### Example 4

Purification of Recombinant Human V165-CTP-V165 Protein

Recombinant human V165-CTP-V165 protein can be purified from the culture medium, such as affinity chromatography. In other embodiments, conventional techniques known to those skilled in the art can be used, including ammonium sulfate precipitation followed by DEAE-Sepharose column chromatography, acid extraction, gel filtration, anion or cation exchange chromatography, DEAE-Sepharose column chromatography, hydroxylapatite chromatography, lectin chromatography, solvent-solvent extraction, ultrafiltration, and HPLC. The purified recombinant protein from the culture medium would be further concentrated by such techniques as, for example, ultrafiltration and tangential flow filtration. In some embodiments where the protein is not secreted into the culture medium, mammalian cells can be lysed, for example, by sonication or chemical treatment, and the homogenate centrifuged to remove cell debris. The supernatant can then be subjected to purification procedures.

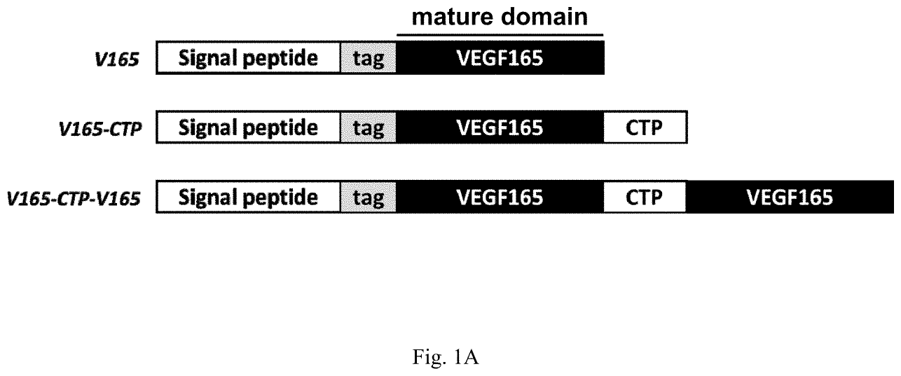

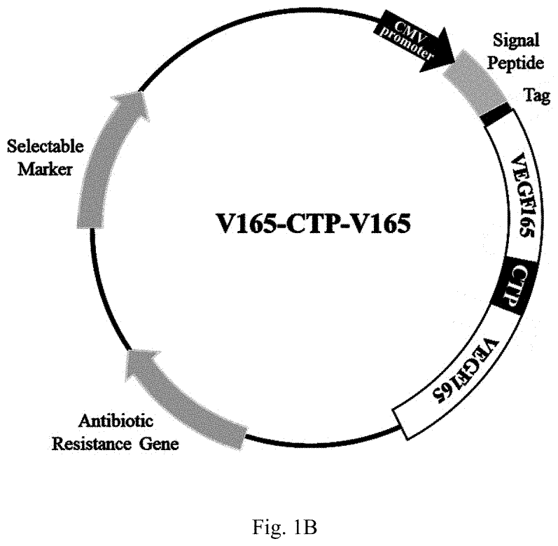

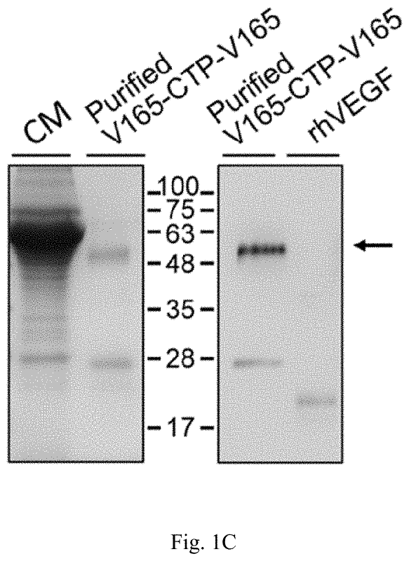

The eluted product was verified by both Coomassie blue staining (Left) and immunoblotting (Right) against human VEGF (FIG. 1D).

### Example 5

The Pharmaceutical Kinetic Assay of rhVEGF, V165-CTP, and V165-CTP-V165 was Determined by Using Luciferase Reporter Assay

To demonstrate the efficacy of the purified recombinant proteins, HEK-293T cells with VEGF type 2 receptor (VEGFR2) receptor and NFAT reporter were treated with purified V165-CTP (VEGFCTP), single-chain VEGF165 (V165-CTP-V165), or human VEGF protein purchased from R&D Systems. Protein efficacy was determined by the luciferase activity.

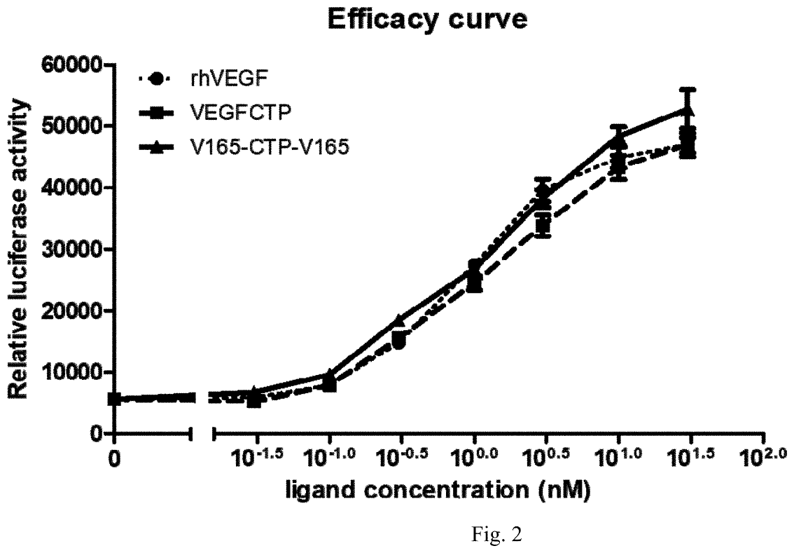

This pharmaceutical kinetic assay indicated that single-chain VEGF165 induced downstream NFAT reporter activity as strong as its natural form, suggesting that they exhibit similar affinity and efficacy to receptors (FIG. 2).

The term “VEGF receptor” or “VEGFR” referring the cell-surface receptor for VEGF, which includes different types that correspond to the VEGF variants.

### Example 6

The Single-Chain VEGF165 (V165-CTP-V165) Shows Prolonged Physiological Stability In Vitro and In Vivo

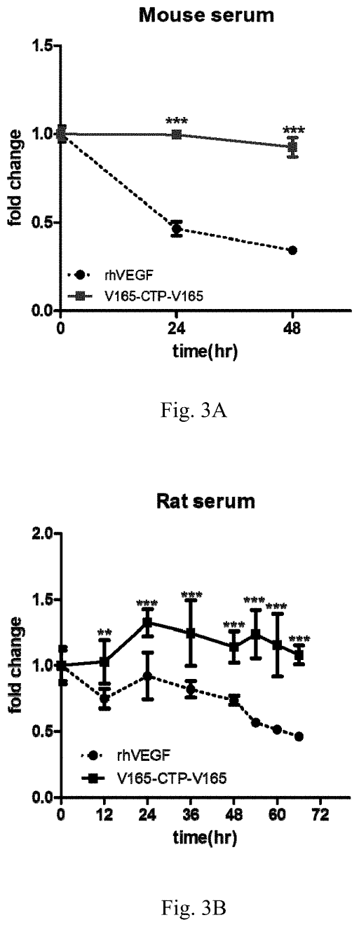

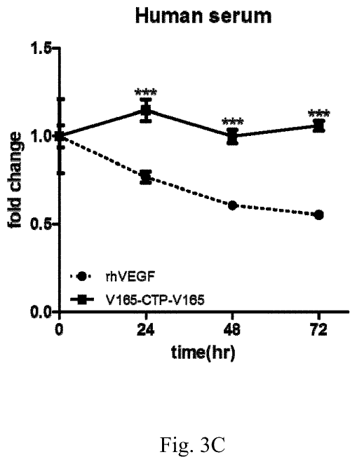

In vitro stability assay was here used to measure the degradation of the recombinant proteins when co-incubated with serum. As shown in FIG. 3A, the in vitro stability of single-chain VEGF165 in mouse serum was much longer than that of commercial VEGF in the tested period. Similar results were found in the test of rat serum (FIG. 3B). Most importantly, single-chain VEGF165 showed a slow degradation rate in human serum (FIG. 3C).

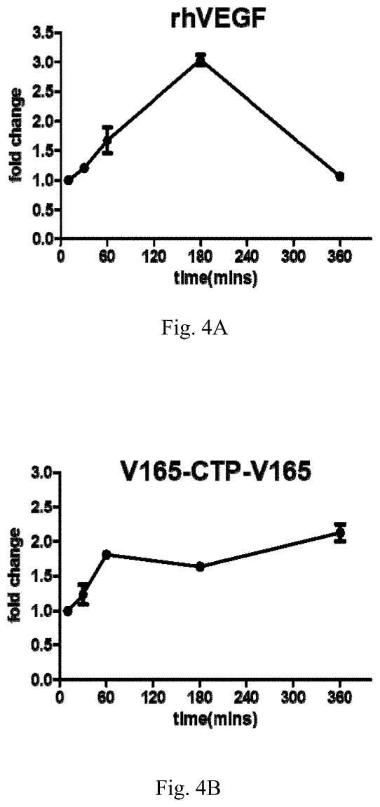

Due to the increase in vitro stability, which might reflect the change in protein in vivo longevity, the invention was thereby performed in vivo injection to determine the protein circulatory half-life. Commercial rhVEGF (R&D) or the single-chain VEGF165 was injected intravenously into 6-8-week-old ICR mice. At indicated time intervals, the blood was drawn from the above-mentioned ICR mice followed by serum preparation. The half-life of tested VEGF in serum was monitored by ELISA. The results showed that a relatively high level of the single-chain VEGF165 was still detectable in serum after 6 hours (FIG. 4B). Nevertheless, the serum sample with the injection of the commercial VEGF reached the basal level after 6 hours (FIG. 4A). Thus, these results suggest that single-chain VEGF165 (V165-CTP-V165) has prolonged protein duration than commercial VEGF (R&D).

### Example 7

Single-Chain VEGF165 (V165-CTP-V165) Induces Cell Recruitment and Neovascularization In Vivo

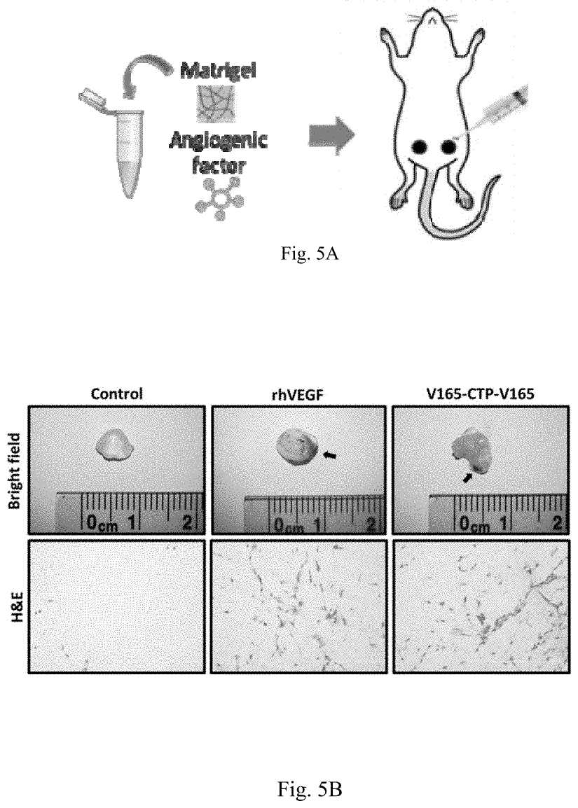

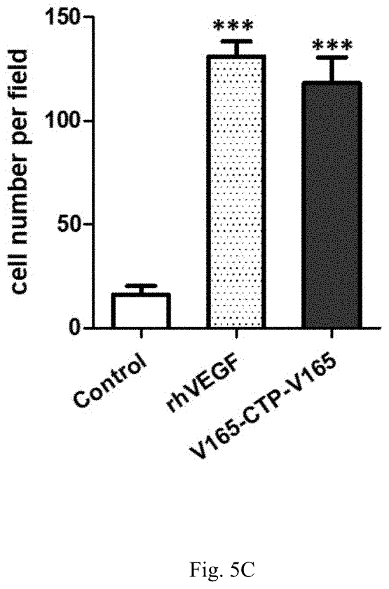

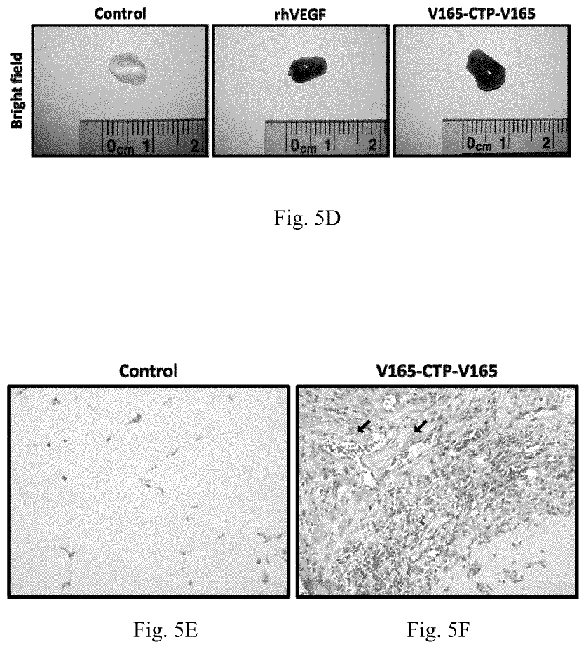

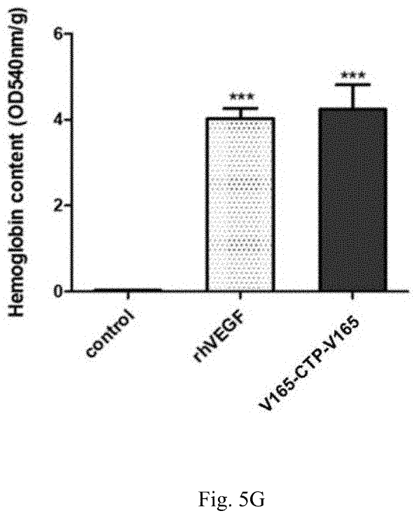

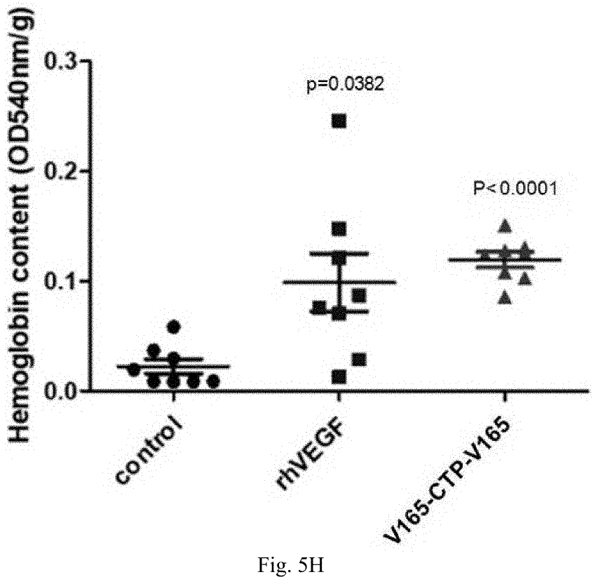

Further, the present embodiment mimicked the physiological environment of cell-matrix to investigate the effect of the angiogenic compounds via verifying the angiogenic effect performed by Matrigel plug assay in vivo. Mice were injected subcutaneously with Matrigel containing recombinant proteins, and the plugs were harvested on the indicated day (FIG. 5A). After 7 days of incubation, the embodiment demonstrated that in the presence of single-chain VEGF165, the Matrigel plug appeared a bit red (as indicated by the arrow in FIG. 5B). This result showed that single-chain VEGF165 is able to recruit the mouse endothelial cells and hematopoietic cells infiltrating into the plug, as observed from the presence of the VEGF purchased from R&D. The degrees of cell infiltration were quantified by the numbers of cells per field (FIG. 5C). Besides, the results of incubation for 14 days showed VEGF-induced neovascularization. Both single-chain VEGF165 (V165-CTP-V165) and commercial VEGF induced functional neo-vessels development into the Matrigel plugs by showing a significant dark red appearance (FIG. 5D), blood vessel formation (FIG. 5E-5F), and increase in hemoglobin contents (FIG. 5G). These neovascularization effects of commercial VEGF and single-chain VEGF165 (V165-CTP-V165), such as increased hemoglobin contents (FIG. 5H), are reproducible in independent experiments.

### Example 8

Single-Chain VEGF165 Promotes Proliferation, Migration, and Tube Formation of Endothelial Cells During the Angiogenesis Process

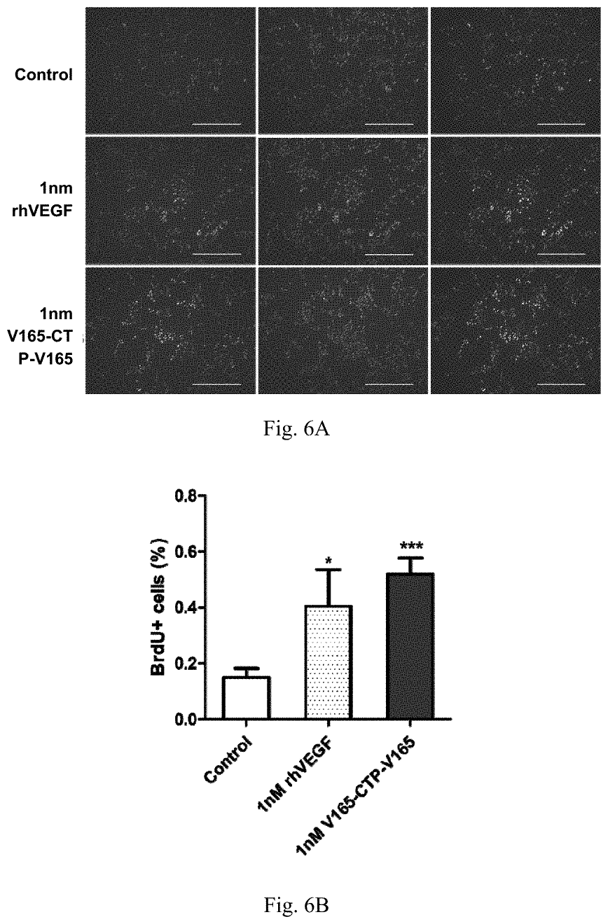

The embodiment explored the bioactivity of single-chain VEGF165 via performing the rates of BrdU incorporation, in vitro wound healing assay, and tube formation assay to evaluate the endothelial cells proliferated, migrated, and invaded the surrounding tissues to form capillary tubules during the angiogenesis process. The level of cells proliferation was measured by the rates of BrdU incorporation, showing a corresponding mitogenesis degree between rhVEGF and single-chain VEGF165 (FIG. 6A-6B).

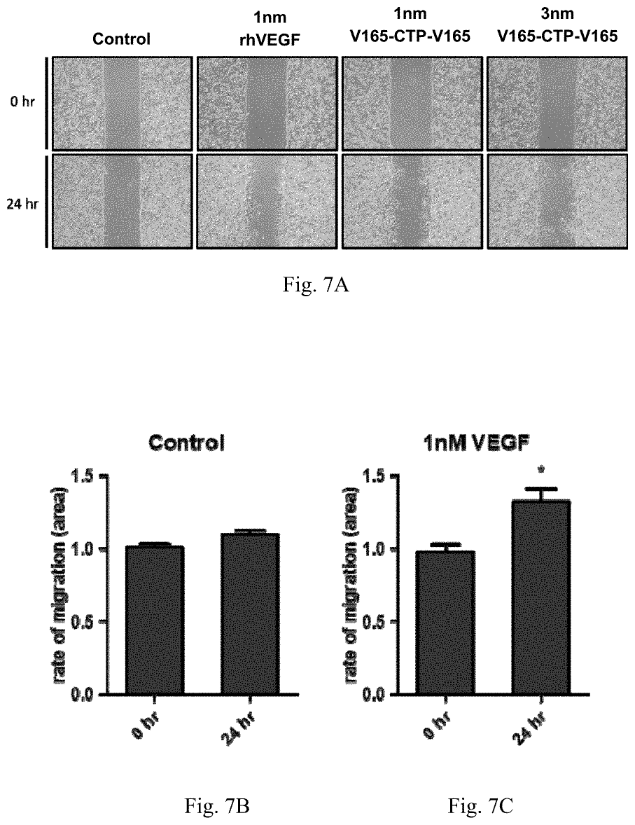

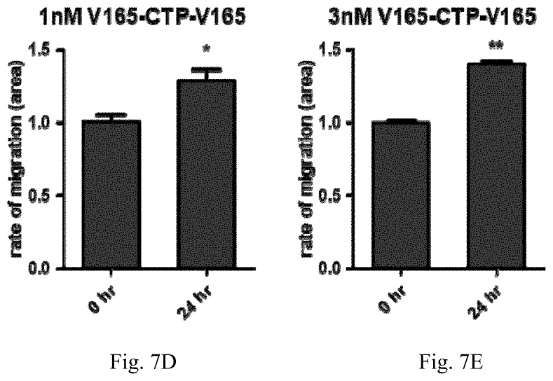

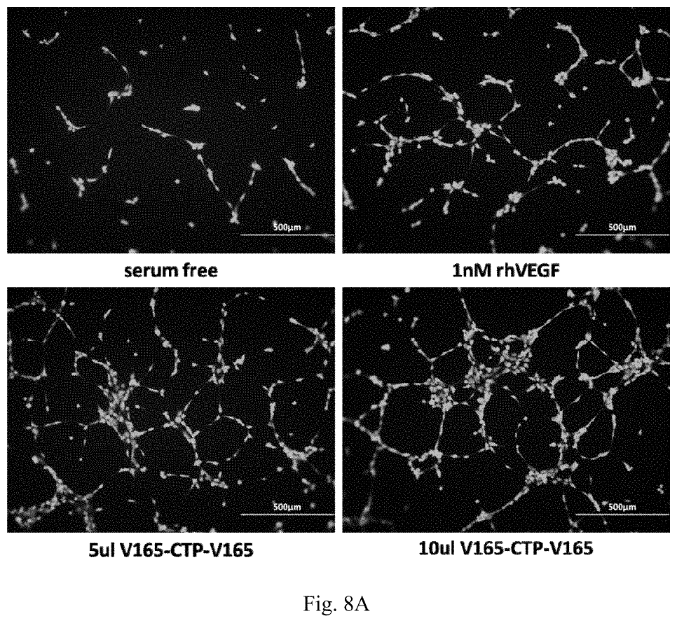

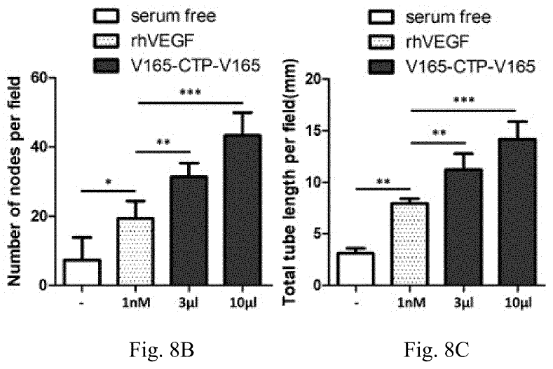

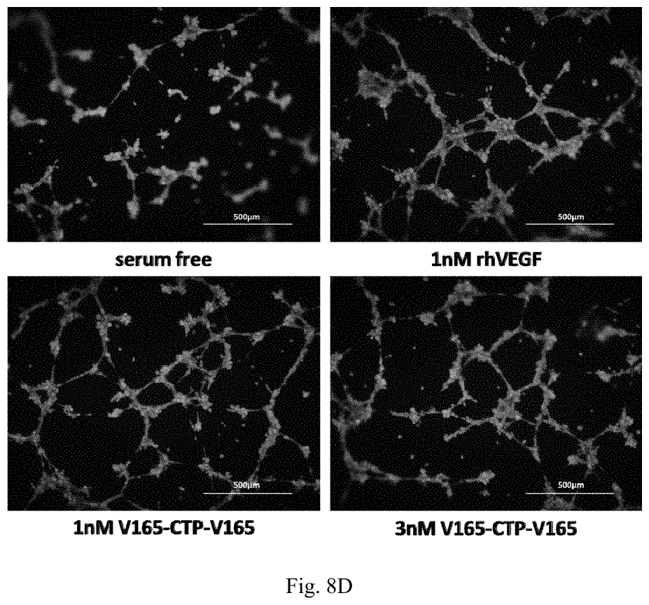

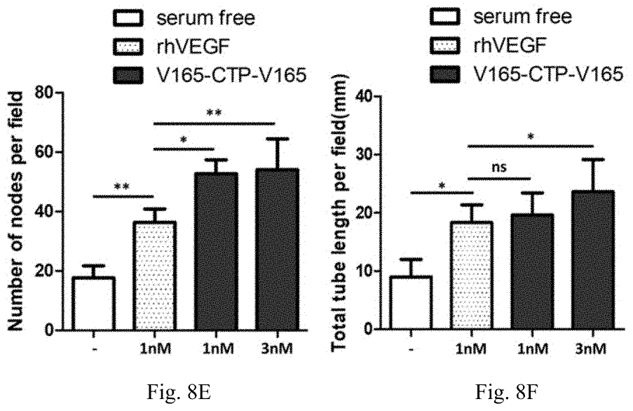

On the other hand, the cell migration effect of single-chain VEGF165 was evaluated by in vitro wound healing assay. Representative photomicrographs, as well as the statistical results, were shown in FIG. 7A and FIG. 7B-7E, respectively. Meanwhile, the results demonstrated that single-chain VEGF165 caused an equivalent degree in the tube formation and branching to human VEGF protein purchased from R&D Systems in human umbilical vein endothelial cells (HUVECs) (FIG. 8A-8C) and mouse microvascular endothelial cells (HMEC1) (FIG. 8D-8F).

### Example 9

Single-Chain VEGF165 (V165-CTP-V165) Enhances Wound Healing Process In Vivo

Prolong of the protein longevity might increase its biopotency in vivo, which is valuable for clinical application. According to the functionality of VEGF, the present invention established excisional wounds in mice to test how the recombinant proteins topically promote angiogenesis to accelerate wound healing procedures at sites of tissue injury.

Mouse excisional wound model was used for in vivo wound healing assay. Hairs on the dorsal skin of 10-12-week-old ICR mice were firstly cut by the hair clipper and then totally depilated by hair removal cream 3 days before the surgery. On the day of surgery, mice were anesthetized by pentobarbital. The dorsal skin of the chest was pulled from the midline with fingers, folded, and punched through both layers with a 4-mm-diameter sterile biopsy punch to create two symmetrical full-thickness excisional wounds besides the midline. After that, 10 μl volume of sterile phosphate-buffered saline (PBS) vehicle, rhVEGF (R&D, AF-293-NA) or V165-CTP-V165 was placed into the wound bed at a single dose of 100 ng per wound. After standing for minutes until the solution was absorbed entirely, wounds administrated with PBS, VEGF, or V165-CTP-V165 were covered with a Tegaderm transparent dressing. Digital photographs were taken on day 0, day 4, and day 8. The wound area was calculated as a percent area of the original wound size; the diameter of the wound was calculated by a graduated scale.

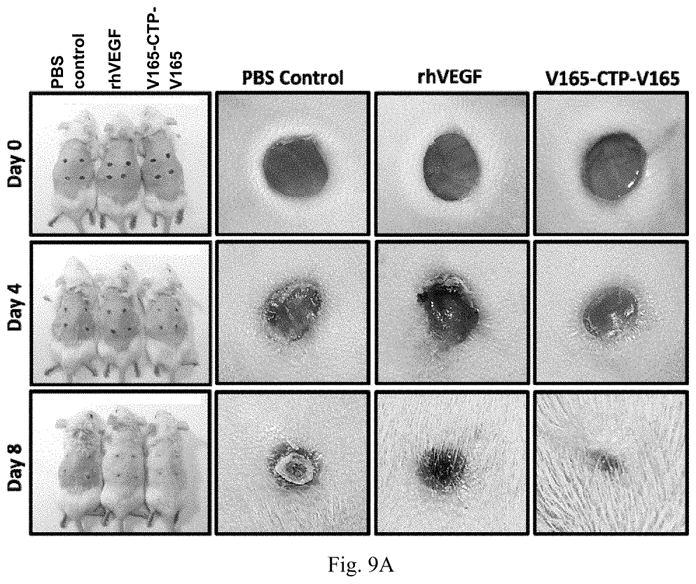

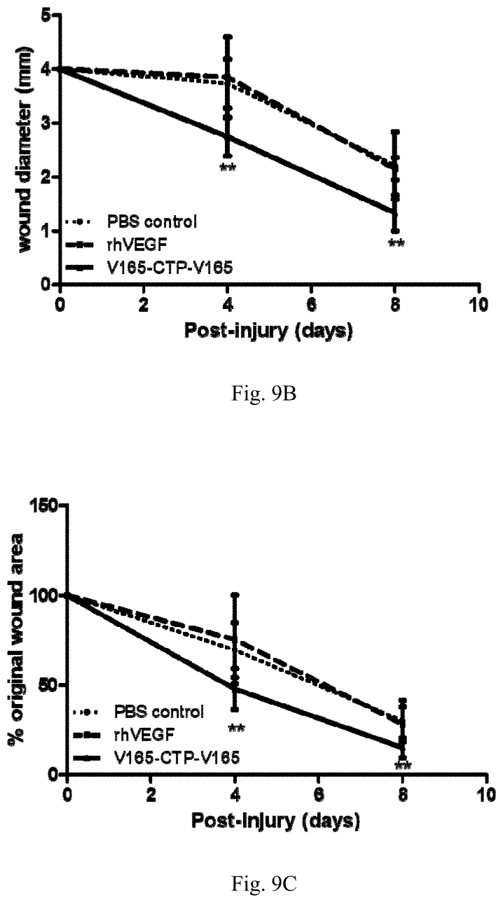

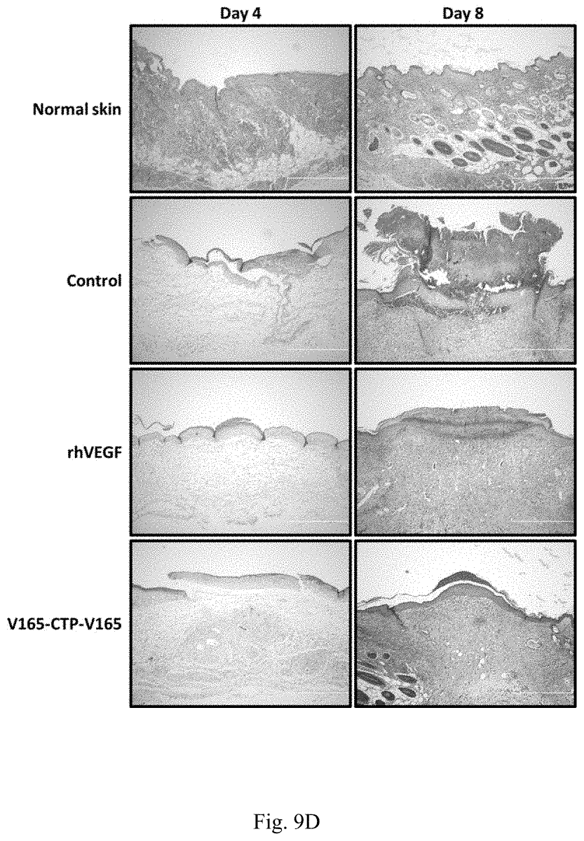

As demonstrated in FIG. 9A, the sizes of wounds in the single-chain VEGF165-treated mice were decreased significantly at day 4 and day 8 compared with the mice treated with commercial VEGF (R&D). The result demonstrated nonsignificant effects on commercial VEGF-treated mice compared with PBS control. As previous studies described, multiple administrations of VEGF were required in order to reach the therapeutic effect for topical medication due to its short half-life. However, single-dose administration of single-chain VEGF165 to wounds resulted in accelerated wound closure compared with VEGF purchased from the R&D Systems (FIG. 9B-9C). Moreover, new hairs were observed to be forming around the wound area in the single-chain VEGF165 treated mice. This phenomenon indicated that the wound healing process had undergone into maturation phase, which occurs collagen reorganization, new follicle formation, and new hair growth.

To further investigate the stage of the wounds, the skin tissues were collected to perform histological analysis (FIG. 9D). The granulation tissue and red blood cells were still present in the PBS-treated wound (FIG. 9D, control). As for the commercial VEGF-treated wound, granulation tissue still appeared, yet re-epithelialization has occurred (FIG. 9D, rhVEGF). Here, the images of single-chain VEGF165-treated wound demonstrated a rapid skin reconstruction, in which the stage of epithelialization and matrix reorganization has completed, and the wound site progresses into the restoration of stratum corneum (FIG. 9D, V165-CTP-V165).

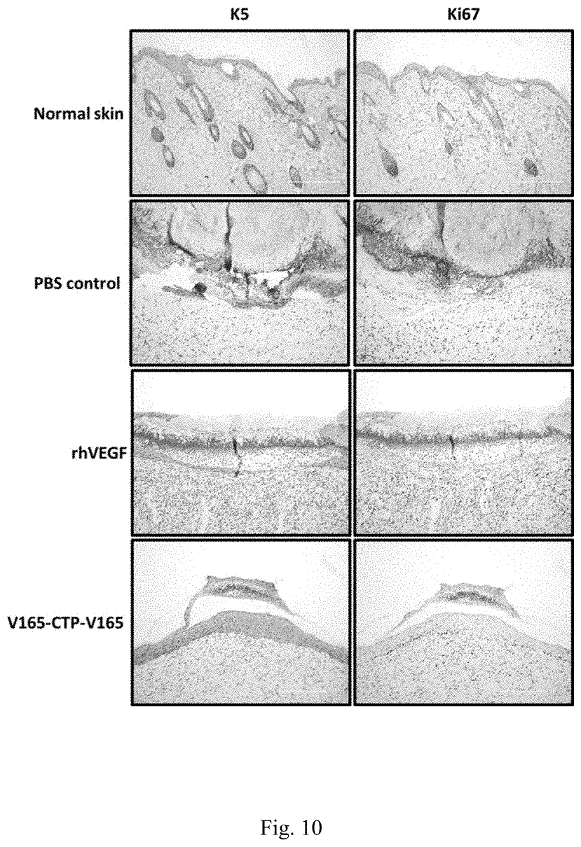

Immunohistochemistry for keratin 5 (K5) detected epithelial cells was revalidated, showing an intense epidermis on the single-chain VEGF165-treated wound (FIG. 10). Besides, Ki67 expression indicated the activation of epidermal stem cells (FIG. 10). An increased Ki67 expression for proliferation was also found in dermis after single-chain VEGF treatment. Overall, the present invention suggests single-chain VEGF165 (V165-CTP-V165) has a notable curative effect for topical medicine, which is likely contributed by the prolonged stability and high biopotency of the protein.

The foregoing detailed description and examples have been provided for clarity of understanding only. No unnecessary limitations are to be understood therefrom. The invention is not limited to the exact details shown and described; many variations will be apparent to one skilled in the art and are intended to be included within the invention defined by the claims.

What is claimed is:

1. A nucleic acid carrier for producing a fusion protein or a derived mRNA comprising:
a first polynucleotide and a second polynucleotide;
wherein the first polynucleotide is linked to the second polynucleotide by a linker polynucleotide;
wherein the first polynucleotide is selected from a VEGF gene;
wherein the second polynucleotide is selected from a VEGF gene.

2. The nucleic acid carrier for producing the fusion protein or the derived mRNA according to claim 1, wherein the VEGF gene comprises VEGF165 gene (SEQ ID NO: 14), VEGF165b gene (SEQ ID NO: 15), VEGF121 gene (SEQ ID NO: 11), VEGF145 gene (SEQ ID NO: 12), VEGF148 gene (SEQ ID NO: 13), VEGF183 gene (SEQ ID NO: 16), VEGF189 gene (SEQ ID NO: 17), and VEGF206 gene (SEQ ID NO: 18).

3. The nucleic acid carrier for producing the fusion protein or the derived mRNA according to claim 2, wherein a VEGF mRNA is derived from the VEGF gene.

4. The nucleic acid carrier for producing the fusion protein or the derived mRNA according to claim 3, wherein the VEGF mRNA comprises VEGF165 mRNA (SEQ ID NO:21), VEGF165b mRNA, VEGF121 mRNA, VEGF145 mRNA, VEGF148 mRNA, VEGF183 mRNA, VEGF189 mRNA, and VEGF206 mRNA.

5. The nucleic acid carrier for producing the fusion protein or the derived mRNA according to claim 1, wherein the linker polynucleotide is selected from CTP gene (SEQ ID NO: 19).

6. The nucleic acid carrier for producing the fusion protein or the derived mRNA according to claim 5, wherein a CTP mRNA (SEQ ID NO:22) is derived from the CTP gene (SEQ ID NO: 19).

7. The nucleic acid carrier for producing the fusion protein or the derived mRNA according to claim 1, wherein the first polynucleotide is encoding a VEGF polypeptide, wherein the second polynucleotide is encoding a VEGF polypeptide.

8. The nucleic acid carrier for producing the fusion protein or the derived mRNA according to claim 7, wherein the VEGF polypeptide comprises VEGF165 polypeptide (SEQ ID NO: 1), VEGF165b polypeptide (SEQ ID NO: 2), VEGF121 polypeptide (SEQ ID NO: 3), VEGF145 polypeptide (SEQ ID NO: 4), VEGF148 polypeptide (SEQ ID NO: 5), VEGF183 polypeptide (SEQ ID NO: 6), VEGF189 polypeptide (SEQ ID NO: 7), and VEGF206 polypeptide (SEQ ID NO: 8).

9. The nucleic acid carrier for producing the fusion protein or the derived mRNA according to claim 1, wherein the linker polynucleotide is encoding CTP polypeptide (SEQ ID NO: 9).

10. The nucleic acid carrier for producing the fusion protein or the derived mRNA according to claim 1, wherein the fusion protein is selected from V165-CTP-V165 polypeptide (SEQ ID NO: 10).

11. The nucleic acid carrier for producing the fusion protein or the derived mRNA according to claim 1, wherein the derived mRNA is selected from V165-CTP-V165 mRNA (SEQ ID NO: 23).

12. A method of treating a subject having a wound comprising administering to the subject the nucleic acid carrier, the derived mRNA or the fusion protein of claim 1, or a pharmaceutical composition comprising the nucleic acid carrier, the derived mRNA, or the fusion protein of claim 1.

13. A method of claim 12, wherein the wound includes normal wound, chronic wound, diabetic foot ulcer, pressure ulcer, decubitus ulcer, venous ulcer, burn, and surgical wound.

14. A method of treating a subject having ischemic tissue or ischemic heart disease comprising administering to the subject the nucleic acid carrier, the derived mRNA or fusion protein of claim 1, or a pharmaceutical composition comprising the nucleic acid carrier, the derived mRNA, or the fusion protein of claim 1.

15. A method of reducing a wound area, a wound size, a wound depth, or a wound boundary comprising administering to a wound the nucleic acid carrier, the derived mRNA, or the fusion protein of claim 1.
